(19) United States
(12) Patent Application Publication
(10) Pub. No.: US 2015/0160241 A1
(43) Pub. Date: Jun. 11, 2015

(54) GAMMA-AMINOBUTYRIC ACID A RECEPTOR SUBUNIT BIOMARKERS

(71) Applicant: Regents of the University of Minnesota, Minneapolis, MN (US)

(72) Inventor: S. Hossein Fatemi, Minneapolis, MN (US)

(73) Assignee: REGENTS OF THE UNIVERSITY OF MINNESOTA, Minneapolis, MN (US)

(21) Appl. No.: 14/563,725

(22) Filed: Dec. 8, 2014

Related U.S. Application Data
(60) Provisional application No. 61/913,793, filed on Dec. 9, 2013.

(51) Int. Cl. G01N 33/68; C12Q 1/68

(52) U.S. Cl. CPC G01N 33/6893; G01N 33/6845; C12Q 1/6883; G01N 2800/50; C12Q 2600/158; C12Q 2600/118; G01N 2333/47; G01N 2333/70571; G01N 2800/302; G01N 2800/304; G01N 2800/30

(57) ABSTRACT

The invention provides physical diagnostic tests for schizophrenia, bipolar disorder, major depression, and autism.

### CROSS-REFERENCE TO RELATED APPLICATIONS

This patent application claims the benefit of priority of U.S. provisional application Ser. No. 61/913,793, filed Dec. 9, 2013, which application is herein incorporated by reference.

### FEDERAL GRANT SUPPORT

The invention was made with government support under IR01 MH086000-01A2 awarded by the National Institutes of Health. The government has certain rights in the invention.

### BACKGROUND

There are currently no reliable physical diagnostic tests for schizophrenia, bipolar disorder, or major depression. Thus, methods are still needed to aid in the diagnosis of these disorders.

### SUMMARY

The present invention provides in certain embodiments a method of determining the increased likelihood of having or developing schizophrenia in a patient, comprising
(a) obtaining a physiological sample from the patient;
(b) quantifying levels of FMRP protein, mGluR5 protein, and GABA_{A} receptor theta (“GABRθ”) protein in the sample, and comparing the levels to a control sample;
(c) determining that the patient has schizophrenia or an increased likelihood of developing schizophrenia based upon the level of FMRP protein, mGluR5 protein, and GABRθ protein in the sample, wherein a lower level of FMRP protein, mGluR5 protein, and GABRθ protein in the sample as compared to the control indicates that the patient has schizophrenia or has an increased likelihood of developing schizophrenia.

The present invention provides in certain embodiments a method of determining the increased likelihood of having or developing schizophrenia in a patient, comprising
(a) obtaining a physiological sample from the patient;
(b) quantifying levels of mGluR5 mRNA and GABRθ mRNA in the sample, and comparing the levels to a control sample;
(c) determining that the patient has schizophrenia or an increased likelihood of developing schizophrenia based upon the levels of mGluR5 mRNA and GABRθ mRNA in the sample, wherein lower levels of mGluR5 mRNA and GABRθ mRNA in the sample as compared to the control indicates that the patient has schizophrenia or has an increased likelihood of developing schizophrenia.

The present invention provides in certain embodiments a method of determining the increased likelihood of having or developing schizophrenia in a patient, comprising
(a) obtaining a physiological sample from the patient;
(b) quantifying levels of FMRP protein, mGluR5 protein, and GABRθ protein in the sample, and comparing the levels to a control sample;
(c) quantifying levels of mGluR5 mRNA and GABRθ mRNA in the sample, and comparing the levels to a control sample; and
(d) determining that the patient has schizophrenia or an increased likelihood of developing schizophrenia based upon the levels of FMRP protein, mGluR5 protein, and GABRθ protein in the sample, and based upon the levels of mGluR5 mRNA and GABRθ mRNA in the sample, wherein lower levels of FMRP protein, mGluR5 protein, and GABRθ protein in the sample and lower levels of mGluR5 mRNA and GABRθ mRNA in the sample as compared to the control indicates that the patient has schizophrenia or has an increased likelihood of developing schizophrenia. The present invention provides in certain embodiments a method for determining whether a patient has or is predisposed to developing schizophrenia comprising:
(a) transporting a physiological sample from a patient suspected of having or being predisposed to developing schizophrenia to a diagnostic laboratory,
(b) detecting in a physiological sample from the patient, a schizophrenia protein profile and mRNA profile, wherein the schizophrenia profile consists of lower levels of FMRP protein, mGluR5 protein, mGluR5 mRNA, GABRθ protein and GABRθ mRNA as compared to a control sample,
(c) identifying that the patient has or is predisposed to the development of schizophrenia when the patient has the schizophrenia profile, and
(d) providing results regarding whether the patient has schizophrenia.

The present invention provides in certain embodiments a method of determining the increased likelihood of having or developing bipolar disorder in a patient, comprising
(a) obtaining a physiological sample from the patient;
(b) quantifying levels of FMRP protein, mGluR5 protein, GABRθ protein, and GABA_{A} receptor rho2 (“GABRρ2”) protein in the sample, and comparing the levels to a control sample;
(c) determining that the patient has bipolar disorder or an increased likelihood of developing bipolar disorder based upon the levels of FMRP protein, mGluR5 protein, GABRθ protein, and GAB p2 protein in the sample, wherein lower levels of FMRP protein, mGluR5 protein, and GABRθ protein, and a higher levels of GABRρ2 protein in the sample as compared to the control indicates that the patient has bipolar disorder or has an increased likelihood of developing bipolar disorder.

The present invention provides in certain embodiments a method of determining the increased likelihood of having or developing bipolar disorder in a patient, comprising
(a) obtaining a physiological sample from the patient;
(b) quantifying levels of FMRP protein, mGluR5 protein, GABRθ protein, and GABRρ2 protein in the sample, and comparing the levels to a control sample;
(c) quantifying levels of GABroh2 mRNA in the sample, and comparing the levels to a control sample;
(d) determining that the patient has bipolar disorder or an increased likelihood of developing bipolar disorder based upon the levels of FMRP protein, mGluR5 protein, GABRθ protein, and GABRρ2 protein in the sample, wherein lower levels of FMRP protein, mGluR5 protein, and GABRθ protein, and a higher levels of GABRρ2 protein and GABRρ2 mRNA in the sample as compared to the control indicates that the patient has bipolar disorder or has an increased likelihood of developing bipolar disorder.

The present invention provides in certain embodiments a method for determining whether a patient has or is predisposed to developing bipolar disorder comprising:
(a) transporting a physiological sample from a patient suspected of having or being predisposed to developing bipolar disorder to a diagnostic laboratory,
(b) detecting in a physiological sample from the patient, a bipolar disorder protein and mRNA profile, wherein the profile consists of lower levels of FMRP protein, mGluR5 protein, GABRθ protein, and an higher levels of GABRρ2 protein and GABroh2 mRNA as compared to a control sample,
(c) identifying that the patient has or is predisposed to the development of bipolar disorder when the patient has the bipolar disorder profile, and
(d) providing results regarding whether the patient has bipolar disorder. The present invention provides in certain embodiments a method of determining the increased likelihood of having or developing major depressive disorder (MDD) in a patient, comprising
(a) obtaining a physiological sample from the patient;
(b) quantifying levels of FMRP protein, GABRθ protein and GABRρ2 protein in the sample, and comparing the levels to a control sample;
(c) determining that the patient has MDD or an increased likelihood of developing MDD based upon the levels of FMRP protein, GABRθ protein, and GABRρ2 protein in the sample, wherein lower levels of FMRP protein and GABRθ protein, and a higher level of GABRρ2protein in the sample as compared to the control indicates that the patient has MDD or has an increased likelihood of developing MDD.

The present invention provides in certain embodiments a method of determining the increased likelihood of having or developing major depressive disorder (MDD) in a patient, comprising
(a) obtaining a physiological sample from the patient;
(b) quantifying levels of FMRP protein, GABRθ protein and GABRρ2 protein in the sample, and comparing the levels to a control sample;
(c) quantifying the level of mGluR5 mRNA in the sample, and comparing the level to a control sample;
(d) determining that the patient has MDD or an increased likelihood of developing MDD based upon the level of FMRP protein, GABRθ protein, and GABRρ2 protein in the sample, wherein lower levels of FMRP protein, GABRθ protein, and mGluR5 mRNA, and higher levels of GABRρ2 protein in the sample as compared to the control indicates that the patient has MDD or has an increased likelihood of developing MDD.

The present invention provides in certain embodiments a method for determining whether a patient has or is predisposed to developing major depressive disorder (MDD) comprising:
(a) transporting a physiological sample from a patient suspected of having or being predisposed to developing MDD to a diagnostic laboratory,
(b) detecting in a physiological sample from the patient, a MDD protein and mRNA profile, wherein the profile consists of decreased levels of FMRP protein, mGluR5 protein, mGluR5 mRNA, and GABRθ protein and GABRθ mRNA as compared to a control sample,
(c) identifying that the patient has or is predisposed to the development of MDD when the patient has the MDD profile, and
(d) providing results regarding whether the patient has MDD.

The present invention provides in certain embodiments a method of determining the increased likelihood of having or developing autism in a patient, comprising:
(a) obtaining a physiological sample from the patient;
(b) quantifying levels of GABRρ2 protein and GABRθ mRNA in the sample, and comparing the levels to a control protein sample;
(c) determining that the patient has autism or an increased likelihood of developing autism based upon the level of GABRρ2 protein in the sample and based on the level of GABRθ mRNA, wherein a lower level of GABRρ2 protein and a lower level of GABRθ mRNA in the sample as compared to the control indicates that the patient has autism or has an increased likelihood of developing autism.

In certain embodiments, the physiological sample is brain tissue, blood (including blood fractions, such as serum) or cerebral spinal fluid (CSF). In certain embodiments, the amino acid level is determined by means of a Western blot. In certain embodiments, the mRNA level is determined by means of quantitative real-time polymerase chain reaction (qRT-PCR).

The term “biomarker” is generally defined herein as a biological indicator, such as a particular molecular feature, that may affect or be related to diagnosing or predicting an individual's health.

“Oligonucleotide probe” can refer to a nucleic acid segment, such as a primer, that is useful to amplify a sequence in the gene of interest that is complementary to, and hybridizes specifically to, a particular sequence in the gene of interest.

As used herein, the term “nucleic acid” and “polynucleotide” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

In one embodiment of the present invention, the method also involves contacting the sample with at least one oligonucleotide probe to form a hybridized nucleic acid and amplifying the hybridized nucleic acid. “Amplifying” utilizes methods such as the polymerase chain reaction (PCR), ligation amplification (or ligase chain reaction, LCR), strand displacement amplification, nucleic acid sequence-based amplification, and amplification methods based on the use of Q-beta replicase. These methods are well known and widely practiced in the art. Reagents and hardware for conducting PCR are commercially available. In another embodiment of the present invention, at least one oligonucleotide probe is immobilized on a solid surface.

### BRIEF DESCRIPTION OF TILE DRAWINGS AND TABLES

FIG. 1. Expression of GABRθ/β-actin (a), GABRρ2/β-actin (b), mGluR5 dimer/β-actin (c), mGluR5 monomer/β-actin (d), FMRP/β-actin (e), and β-actin (f) in lateral cerebella of healthy controls vs. subjects with bipolar disorder, major depressive disorder, and schizophrenia. Histogram bars shown as mean±standard error, .*, p<0.05. FMRP and β-actin data reprinted from Schizophrenia Research, 124(1-3):246-247, Fatemi, S. H., Kneeland, R. E., Liesch, S. B., Folsom, T. D., Fragile X mental retardation protein levels are decreased in major psychiatric disorders, page 247, FIG. 1, Copyright (2010), with permission from Elsevier.

FIG. 2. Expression of GABRθ/NSE (a), GABRρ2/NSE (b), mGluR5 dimer/NSE (c), mGluR5 monomer/NSE (d), FMRP/NSE (e), and NSE (f) in lateral cerebella of healthy controls vs. subjects with bipolar disorder, major depressive disorder, and schizophrenia. Histogram bars shown as mean±standard error, *, p<0.05.

FIG. 3. Expression of GABRθ/β-actin (a), GABRρ2/13-actin (b), mGluR5 dimer/β-actin (c), mGluR5 monomer/β-actin (d), FMRP/β-actin (e), and β-actin (f) in BA9 of healthy controls vs. subjects with bipolar disorder, and schizophrenia. Histogram bars shown as mean±standard error, *, p<0.05.

FIG. 4. Expression of GABRθ/NSE (a), GABRρ2/NSE (b), mGluR5 dimer/NSE (c), mGluR5 monomer/NSE (d), FMRP/NSE (e), and NSE (f) in BA9 of healthy controls vs. subjects with bipolar disorder, and schizophrenia. Histogram bars shown as mean±standard error, *, p<0.05.

FIG. 5. Summary of mRNA and protein expression for GABRθ, GABRρ2, mGluR5, and FMRP in lateral cerebella and BA9 of subjects with schizophrenia. Concordant results for mRNA and protein were obtained for GABRθ and mGluR5 in lateral cerebellum. Decreased expression of GABRθ protein in BA9 may lead to a positive feedback loop increasing mRNA expression. Protein levels for mGluR5 and FMRP were reduced significantly in both brain sets. ↑, increased expression; ↓, reduced expression, --, no change.

FIG. 6. Summary of mRNA and protein expression for GABRθ, GABRρ2, mGluR5, and FMRP in lateral cerebella and BA9 of subjects with bipolar disorder. Concordant results for mRNA and protein were obtained for GABRρ2 in lateral cerebellum. Decreased expression of GABRθ protein in lateral cerebellum may lead to a positive feedback loop increasing mRNA expression. Protein levels for GABRθ, mGluR5, and FMRP were decreased significantly in both brain sites. ↑, increased expression; ↓, reduced expression, --, no change.

FIG. 7. Summary of mRNA and protein expression for GABRθ, GABRρ2, mGluR5, and FMRP in lateral cerebella of subjects with major depression. There were no concordant results in subjects with major depression. However, protein levels for GABRθ and FMRP were reduced significantly while increased for GABRρ2 in major depression. ↑, increased expression; ↓, reduced expression, --, no change.

FIG. 8. Summary of relationships between GABRθ, GABRρ2, mGluR5, and FMRP in three major psychiatric disorders: schizophrenia, bipolar disorder, and major depression. While there are clear biochemical connections between FMRP, mGluR5, and GABRρ2; no direct relationship can be established between GABRθ and FMRP. ↑, increased expression; ↓, reduced expression, --, no change.

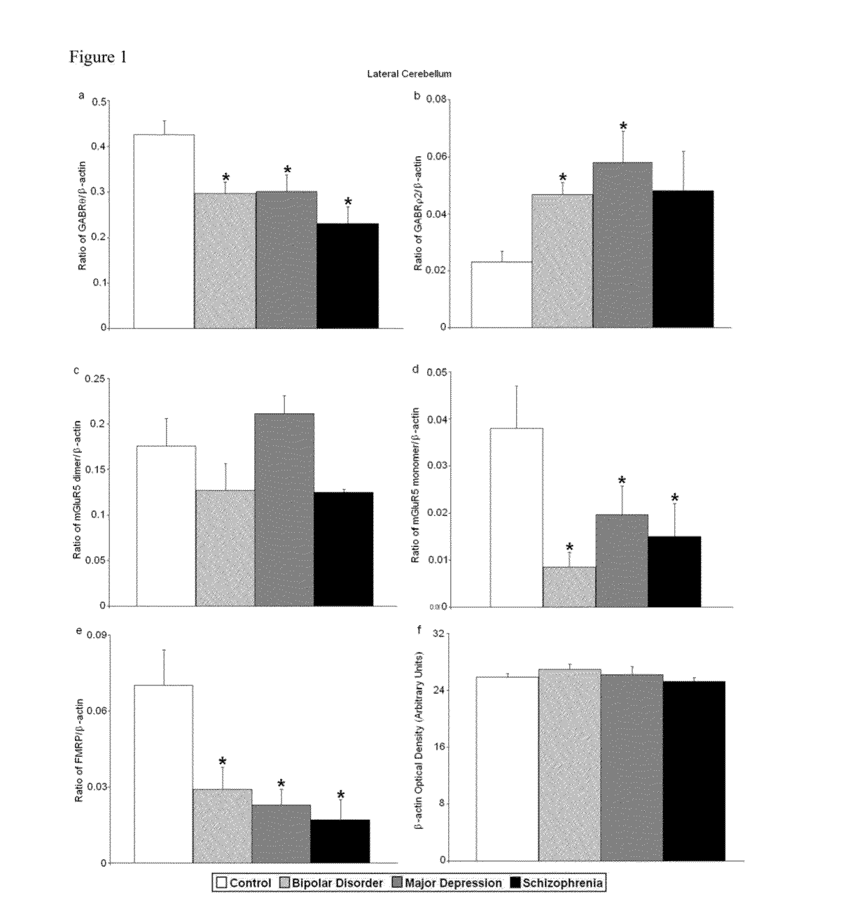

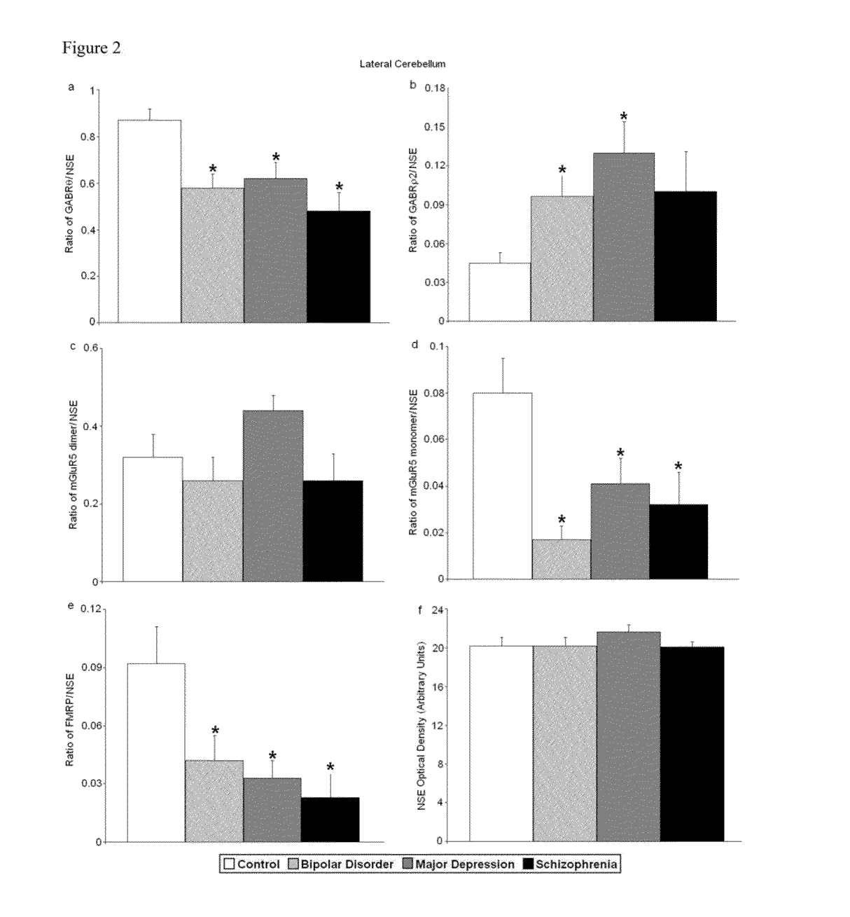

Table 1. Demographic Information for the Four Diagnostic Groups from Stanley Medical Research Institute.

Table 2. Demographic Information for the Three Diagnostic Groups from the McLean 74 Cohort.

Table 3. Western Blotting Results for FMRP, GABRθ, mGluR5, GABRρ2 Values Expressed as Ratios to β-actin and Neuronal Specific Enolase in Lateral Cerebella.

Table 4. Western Blotting Results for FMRP, GABRθ, mGluR5, GABRρ2 Values Expressed as Ratios to β-actin and Neuronal Specific Enolase in BA9.

Table 5. qRT-PCR results for GABRQ, GABRR2, GRM5, and FMR1 in lateral cerebella and BA9 of subjects with schizophrenia and mood disorders.

### DETAILED DESCRIPTION

GABA(A) receptor theta and GABA(A) receptor rho 2 are novel GABA_{A} receptor subunits that we have recently found to have dysfuntional expression in subjects with bipolar disorder; schizophrenia; and major depression. They represent unique biomarkers for these disorders and may serve as potential diagnostic markers. This can be used as a test using brain tissue, blood (or blood product, such as serum) or cerebral spinal fluid to help confirm diagnoses of major psychiatric disorders.

Nucleic Acid Amplification Methods

According to the methods of the present invention, the amplification of DNA present in a physiological sample may be carried out by any means known to the art. Examples of suitable amplification techniques include, but are not limited to, polymerase chain reaction (including, for RNA amplification, reverse-transcriptase polymerase chain reaction), ligase chain reaction, strand displacement amplification, transcription-based amplification, self-sustained sequence replication (or “3SR”), the Qβ replicase system, nucleic acid sequence-based amplification (or “NASBA”), the repair chain reaction (or “RCR”), and boomerang DNA amplification (or “BDA”).

The bases incorporated into the amplification product may be natural or modified bases (modified before or after amplification), and the bases may be selected to optimize subsequent electrochemical detection steps.

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized that is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe of the present invention), the probe carrying a detectable label, and then detecting the label in accordance with known techniques. Where the nucleic acid to be amplified is RNA, amplification may be carried out by initial conversion to DNA by reverse transcriptase in accordance with known techniques. Strand displacement amplification (SDA) may be carried out in accordance with known techniques. For example, SDA may be carried out with a single amplification primer or a pair of amplification primers, with exponential amplification being achieved with the latter. In general, SDA amplification primers comprise, in the 5′ to 3′ direction, a flanking sequence (the DNA sequence of which is noncritical), a restriction site for the restriction enzyme employed in the reaction, and an oligonucleotide sequence (e.g., an oligonucleotide probe of the present invention) that hybridizes to the target sequence to be amplified and/or detected. The flanking sequence, which serves to facilitate binding of the restriction enzyme to the recognition site and provides a DNA polymerase priming site after the restriction site has been nicked, is about 15 to 20 nucleotides in length in one embodiment. The restriction site is functional in the SDA reaction. The oligonucleotide probe portion is about 13 to 15 nucleotides in length in one embodiment of the invention.

Ligase chain reaction (LCR) is also carried out in accordance with known techniques. In general, the reaction is carried out with two pairs of oligonucleotide probes: one pair binds to one strand of the sequence to be detected; the other pair binds to the other strand of the sequence to be detected. Each pair together completely overlaps the strand to which it corresponds. The reaction is carried out by, first, denaturing (e.g., separating) the strands of the sequence to be detected, then reacting the strands with the two pairs of oligonucleotide probes in the presence of a heat stable ligase so that each pair of oligonucleotide probes is ligated together, then separating the reaction product, and then cyclically repeating the process until the sequence has been amplified to the desired degree. Detection may then be carried out in like manner as described above with respect to PCR.

Diagnostic techniques that are useful in the methods of the invention include, but are not limited to direct DNA sequencing, PFGE analysis, allele-specific oligonucleotide (ASO), dot blot analysis and denaturing gradient gel electrophoresis, and are well known to the artisan.

Nucleic acid analysis via microchip technology is also applicable to the present invention.

Oligonucleotide Probes As noted above, the methods of the present invention is useful for detecting the level of a protein or mRNA in a sample.

Oligonucleotide probes may be prepared having any of a wide variety of base sequences according to techniques that are well known in the art. Suitable bases for preparing the oligonucleotide probe may be selected from naturally occurring nucleotide bases such as adenine, cytosine, guanine, uracil, and thymine; and non-naturally occurring or “synthetic” nucleotide bases such as 7-deaza-guanine 8-oxo-guanine, 6-mercaptoguanine, 4-acetylcytidine, 5-(carboxyhydroxyethyl)uridine, 2′-O-methylcytidine, 5-carboxymethylamino-methyl-2-thioridine, 5-carboxymethylaminomethyluridine, dihydrouridine, 2′-O-methylpseudouridine, β,D-galactosylqueosine, 2′-O-methylguanosine, inosine, N6-isopentenyladenosine, 1-methyladenosine, 1-methylpseeudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylamninomethyluridine, 5-methoxyaminomethyl-2-thiouridine, β,D-mannosylqueosine, 5-methloxycarbonylmethyluridine, 5-methoxyuridine, 2-methyltio-N6-isopentenyladenosine, N-(9-β-D-ribofuranosyl-2-methylthiopurine-6-yl)carbamoyl)threonine, N-((9-β-D-ribofuranosylpurine-6-yl)N-methyl-carbamoyl)threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-Methylurdine, N-((9-beta-D-ribofuranosylpurine-6-yl)carbamoyl)threonine, 2′-O-methyl-5-methyluridine, 2′-O-methylurdine, wybutosine, and 3-β-amino-3-carboxypropyl)uridine. Any oligonucleotide backbone may be employed, including DNA, RNA (although RNA is less preferred than DNA), modified sugars such as carbocycles, and sugars containing 2′ substitutions such as fluoro and methoxy. The oligonucleotides may be oligonucleotides wherein at least one, or all, of the intemucleotide bridging phosphate residues are modified phosphates, such as methyl phosphonates, methyl phosphonotlioates, phosphoroinorpholidates, phosphoropiperazidates and phosplioramidates (for example, every other one of the intemucleotide bridging phosphate residues may be modified as described). The oligonucleotide may be a “peptide nucleic acid” such as described in Nielsen et al., Science, 254, 1497-1500 (1991).

The only requirement is that the oligonucleotide probe should possess a sequence at least a portion of which is capable of binding to a known portion of the sequence of the DNA sample.

It may be desirable in some applications to contact the DNA sample with a number of oligonucleotide probes having different base sequences (e.g., where there are two or more target nucleic acids in the sample, or where a single target nucleic acid is hybridized to two or more probes in a “sandwich” assay).

The nucleic acid probes provided by the present invention are useful for a number of purposes. The probes can be used to detect PCR amplification products.

Hybridization Methodology

The DNA (or nucleic acid) sample may be contacted with the oligonucleotide probe in any suitable manner known to those skilled in the art. For example, the DNA sample may be solubilized in solution, and contacted with the oligonucleotide probe by solubilizing the oligonucleotide probe in solution with the DNA sample under conditions that permit hybridization. Suitable conditions are well known to those skilled in the art. Alternatively, the DNA sample may be solubilized in solution with the oligonucleotide probe immobilized on a solid support, whereby the DNA sample may be contacted with the oligonucleotide probe by immersing the solid support having the oligonucleotide probe immobilized thereon in the solution containing the DNA sample.

Positron Emission Tomography (PET)

A positron emission tomography (PET) scan is an imaging test that uses a radioactive substance called a tracer.

Molecular imaging technologies are widely used clinical tools for the diagnosis, staging, and monitoring or therapeutic responses. Many different technologies have been developed to image the structure and function of systems such as autoradiography, optical imaging, positron emission tomography (PET), magnetic resonance imaging (MRI), and X-ray computed tomography (CT). Among those, PET is the only non-invasive technology that can measure metabolic, biochemical and functional activity in vivo. Since morphological response to chemotherapy or radiation therapy lags behind the course of the treatment, analysis of PET images can potentially detect pathological features and therapeutic response before they are visible on CT and MRI images, and thus PET is emerging as a valuable clinical tool to monitor therapeutic responses in patients.

PET imaging requires positron emitting radioisotopes such as oxygen (¹⁴O, ¹⁵O), nitrogen (¹³N), fluorine-18 (¹⁸F), and Carbon (¹¹C) incorporated into pharmaceutical probes to observe selective accumulation in a tissue of interest. Two of the most extensively used PET probes are ¹⁸F-fluorodeoxyglucose (¹⁸F-FDG) and ¹⁸F-fluorothymidine (¹⁸F-FLT). The ¹⁸F-FDG probe targets metabolic activity in a non-specific way resulting in high background labeling of normal tissues such as brain and areas of inflammation. In certain embodiments, the present invention provides a method of detection in a cell comprising contacting the compound as described above to the cell, and imaging the cell by means of positron emission tomography (PET), wherein the compound is employed as a tracer. In certain embodiments, the method further comprises the steps of imaging the subject to detect the distribution of the tracer. In certain embodiments, the method further comprises the steps of imaging the subject and analyzing the imaging data. In certain embodiments, the imaging in the subject is performed by positron emission tomography, single photon emission computed tomography or a combination thereof.

The invention will now be illustrated by the following non-limiting Examples.

### Example 1

### mRNA and Protein Expression for Novel GABAA Receptors Θ And P2 are Altered in Schizophrenia and Mood Disorders; Relevance to FMRP-mGluR5 Signaling Pathway

Fragile X mental retardation protein (FMRP) is an RNA binding protein which targets approximately 4% of all mRNAs expressed in brain. Previous work by our laboratory demonstrated significantly lower protein levels for FMRP in lateral cerebella of subjects with schizophrenia, bipolar disorder, and major depression when compared with controls. Absence of FMRP expression in animal models of fragile X syndrome (FXS) has been shown to reduce expression of gamma-amino butyric acid A (GABA_{A}) receptor mRNAs. Previous work by our laboratory has found reduced expression of FMRP as well as multiple GABA_{A} and GABA_{B} receptor subunits in subjects with autism. Less is known about levels for GABA_{A} subunit protein expression in brains of subjects with schizophrenia and mood disorders. In the current study we have expanded our previous studies to examine the protein and mRNA expression of two novel GABA_{A} receptors, theta (GABRθ) and rho 2 (GABRρ2) as well as FMRP, and metabotropic glutamate receptor 5 (mGluR5) in lateral cerebella of subjects with schizophrenia, bipolar disorder, major depression, and healthy controls and in superior frontal cortex [Brodmann Area 9 (BA9)] of subjects with schizophrenia, bipolar disorder, and healthy controls. We observed multiple statistically significant mRNA and protein changes in levels of GABRθ, GABRρ2, mGluR5 and FMRP molecules including concordant reductions in mRNA and proteins for GABRθ and mGluR5 in lateral cerebella of subjects with schizophrenia; for increased mRNA and protein for GABRρ2 in lateral cerebella of subjects with bipolar disorder; and for reduced mRNA and protein for mGluR5 in BA9 of subjects with bipolar disorder. There were no significant effects of confounds on any of the results.

Impairment of the gamma-aminobutyric acid (GABA) signaling system is believed to partially account for behavioral and cognitive deficits associated with schizophrenia and mood disorders. Reduction of GABA_{A} receptor transmission has also been associated with anxiety, panic, impaired learning and memory. GABA_{A} receptors are responsible for mediating the fast inhibitory action of GABA and are important sites for clinical action of a number of drugs including benzodiazepines, barbiturates, and anesthetics. Recent work has suggested that proper GABAergic neurotransmission is required for network oscillations that facilitate processing of information both in and between various brain regions and that this may be required for normal cognition. Altered expression of GABA_{A} receptor subunits could impair these oscillations and result in improper cognitive function. Little is currently known about GABA_{A} receptor subunit expression in schizophrenia and mood disorders although it is likely that changes in GABA_{A} receptor would result in reduced GABAergic transmission.

Recent evidence provides a linkage between GABA neurotransmission and fragile X mental retardation protein (FMRP). FMRP is an RNA binding protein that has been estimated to regulate translation of 842 transcripts in brain. In animal models of fragile X syndrome (FXS) the absence of FMRP is accompanied by reduced mRNA expression of GABA_{A} receptor subunits including alpha 1 (α1), α3, α4, beta 1 (β1), β2, delta (δ), gamma 1 (γ1), and γ2 in frontal cortex, while there was no change in cerebellum. A functional consequence of this reduced expression has been observed in fragile X mental retardation 1 (Fmr1) knockout (KO) mice which display impaired GABAergic signaling in striatal neurons as measured by increased frequency of spontaneous and miniature inhibitory postsynaptic currents and reduced paired pulse ratio of inhibitory postsynaptic currents. FMRP normally represses metabotropic glutamate receptor 5 (mGluR5) signaling while the absence of FMRP has been hypothesized to lead to unregulated mGluR5 signaling and ultimately result in the various abnormal phenotypes associated with FXS. Animal studies-using antagonists of the mGluR5 receptor have rescued learning and behavioral deficits associated with FXS and reduced seizures in FMR1 knockout mice.

Recently, we reported on reduced protein expression of FMRP in lateral cerebellum from subjects with schizophrenia, bipolar disorder, and major depression. (Fatemi S H, Kneeland R E, Liesch S B, Folsom T D. Fragile X mental retardation protein levels are decreased in major psychiatric disorders [letter]. Schizophr Res 2010a;124: 246-247) These results are novel as gene association studies have not identified fragile X mental retardation 1 (FMR1), the gene that codes FMRP, as a candidate gene for schizophrenia. However, a recent study verified our earlier study, finding reduced FMRP expression in peripheral blood lymphocytes from subjects with schizophrenia. Kovács et al. (Kovacs T, Keleman O, Keri S. Decreased fragile X mental retardation protein (FMRP) is associated with lower IQ and earlier illness onset in patients with schizophrenia. Psychiatry Res 2013 (in press)) found that age of onset and IQ predicted FMRP levels but chlorpromazine-equivalent antipsychotic dose did not. Importantly, none of the study subjects showed the CGG triplet expansion which normally causes silencing of the FMR1 gene in subjects with FXS. Combined with our findings of reduced FMRP expression in cerebellar vermis and prefrontal cortex of subjects with autism, who were not comorbid for FXS, reduction of FMRP expression may be a hallmark of multiple psychiatric disorders.

Based on the evidence from animal models that decrease in FMRP expression results in reduced expression of GABA_{A} receptor subunit mRNA and our finding of significantly reduced FMRP in cerebella of subjects with schizophrenia and mood disorders, we hypothesized that we would observe reduced expression of the GABA_{A} receptor subunits in lateral cerebella from the same diagnostic groups. An initial screen of several GABA receptor subunits in lateral cerebella of subjects with schizophrenia, bipolar disorder, and major depression found reductions in GABA_{B} receptor subunits one and two (GABBR1 and GABBR2) (Fatemi S H, Folsom T D, Thuras P D. Deficits in GABA(B) receptor system in schizophrenia and mood disorders: a postmortem study. Schizophr Res 2011b;128: 37-43) Here, we report novel findings regarding alterations in levels of mRNA and protein for GABA_{A} receptor theta (GABRθ) and GABA_{A} receptor rho 2 (GABRρ2) as well as mGluR5 and FMRP levels in lateral cerebellum and BA9 of subjects with schizophrenia and mood disorders. These results demonstrate disruption of the GABAergic and FMRP-mGluR5 signaling systems in subjects with schizophrenia and mood disorders.

Materials and Methods

Brain Procurement. The Institutional Review Board of the University of Minnesota-School of Medicine has approved this study. Postmortem lateral cerebella were obtained from the Stanley Foundation Neuropathology Consortium under approved ethical guidelines. Postmortem superior frontal cortex (BA9) was obtained from the McLean 74 Cohort, Harvard Brain and Tissue Resource Center. DSM-IV diagnoses were established prior to death by neurologists and psychiatrists using information from all available medical records and from family interviews. Details regarding the subject selection, demographics, diagnostic process, and tissue processing were collected by the Stanley Medical Research Foundation and the Harvard Brain and Tissue Resource Center. The Stanley collection consisted of 15 subjects with schizophrenia, 15 subjects with bipolar disorder, 14 with major depression without psychotic features and 14 normal controls (Table 1). The McLean 74 Cohort consists of 20 subjects with schizophrenia, 19 subjects with bipolar disorder, and 29 normal controls (Table 2). All groups were matched for age, sex, race, postmortem interval and hemispheric side.

SDS-PAGE and Western Blotting. Brain tissue was prepared as previously described. (Fatemi S H, Kneeland R E, Liesch S B, Folsom T D. Fragile X mental retardation protein levels are decreased in major psychiatric disorders [letter]. Schizophr Res 2010a;124: 246-247; Fatemi S H, Folsom T D, Kneeland R E, Liesch S B. Metabotropic glutamate receptor 5 upregulation in children with autism is associated with underexpression of both fragile X mental retardation protein and GABAA receptor beta 3 in adults with autism. Anat Rec 2011a;294:1635-1645; Fatemi S H, Folsom T D, Thuras PD. Deficits in GABA(B) receptor system in schizophrenia and mood disorders: a postmortem study. Schizophr Res 2011b;128: 37-43; Fatemi S H, King D P, Reutiman T J, Folsom T D, Laurence J A, Lee S, et al. PDE4B polymorphisms and decreased PDE4B expression are associated with schizophrenia. Schizophr Res 2008;101:36-49; Fatemi S H, Reutiman T J, Folsom T D, Thuras P D. GABA(A) Receptor Downregulation in Brains of Subjects with Autism. J Autism Dev Disord 2009a; 39:233-230; Fatemi S H, Folsom T D, Reutiman T J, Thuras P D. Expression of GABA(B) Receptors Is Altered in Brains of Subjects with Autism. Cerebellum 2009b;8:64-69; Fatemi S H, Reutiman T J, Folsom T D, Rooney R J, Patel D H, Thuras P D. mRNA and protein levels for GABAAalpha4, alpha5, beta1, and GABABR1 receptors are altered in brains of subjects with autism. J Autism Dev Disord 2010b;40:743-750). For lateral cerebellum, 60 μg of tissue was used while for BA9, 30 μg of tissue was used. For mGluR5 and FMRP we used 6% resolving gels while for GABRθ, GABRρ2, neuronal specific enolase (NSE), and beta-actin we used 10% resolving gels.

We minimized interblot variability by including samples from subjects of each group (control, schizophrenia, bipolar disorder, major depression) on each gel. Samples were run in duplicate. Samples were electrophoresed for 15 minutes at 75V followed by 55 minutes at 150V. Samples were then electroblotted onto nitrocellulose membranes for 2 h at 300 mAmp at 4° C. Blots were blocked with 0.2% I-Block (Tropix, Bedford, Mass., USA) in PBS with 0.3% Tween 20 for one hour at room temperature (RT) followed by an overnight incubation in primary antibodies at 4° C. The primary antibodies used were anti-GABRθ (ab49188, Abcam (Cambridge, Mass., USA) 1:1000), anti-GABRρ2 (ab83223, Abcam, 1:500), anti-FMRP (MAB2160, Millipore (Temecula, Calif., USA), 1:500), anti-mGluR5 (ab53090, Abcam, 1:300), anti-NSE (1:2,000; Abcam Inc., Cambridge, Mass., USA), and anti-β actin (A5441, Sigma Aldrich (St. Louis, Mo., USA), 1:5 000). Blots were washed for 30 minutes in PBS supplemented with 0.3% Tween 20 (PBST) for 30 min. at RT and were subsequently incubated in the proper secondary antibodies. Secondary antibodies were goat anti-mouse IgG (A9044, Sigma Aldrich, 1:80 000) and goat anti-rabbit IgG (A9169, Sigma Aldrich, 1:80 000). Blots were washed twice in PBST for 15 min., each. Following the second wash, bands were visualized using the ECL-plus detection system (GE Healthcare, Little Chalfont, Buckinghamshire, UK) and exposed to CL-Xposure film (Thermo Scientific, Rockford, Ill., USA). The molecular weights of approximately 224 kDa (dimer) and 112 kDa (monomer) for mGluR5; 73 kDa (FMRP); 70 kDa (GABRθ); 54 kDa (GABRρ2); 46 kDa (NSE), and 42 kDa (β-actin) immunoreactive bands were quantified with background subtraction using a Bio-Rad GS-800 Calibrated Densitometer (Bio-Rad, Hercules, Calif., USA) and Quantity One 1-D Analysis software (Bio-Rad, Hercules, Calif., USA). Sample densities were analyzed blind to nature of diagnosis. Results obtained are based on at least two independent experiments.

Quantitative Real Time Polymerase Chain Reaction (qRT-PCR). We performed qRT-PCR as previously described (Fatemi S H, Reutiman T J, Folsom T D, Rooney R J, Patel D H, Thuras P D. mRNA and protein levels for GABAAalpha4, alpha5, betal, and GABABR1 receptors are altered in brains of subjects with autism. J Autism Dev Disord 2010b; 40:743-750). Raw data were analyzed using the Sequence Detection Software RQ Manager (ABI, Foster City, Calif.) while relative quantitation using the comparative threshold cycle (C_{T} method) was performed in Bioconductor using the ABqPCR package in Microsoft Excel (ABI Technote#2: Relative Gene Expression Quantitation). Calculations were done assuming that 1 delta Ct equals a 2-fold difference in expression. Significance values were determined using unpaired t-tests. The probe IDs used were: 1) GABA_{A} receptor theta (GABRQ): Hs00610921_ml; 2) GABA_{A} receptor rho 2 (GABRR2): Hs00266703_ml; 3) Fragile X mental retardation 1 (FMR1): Hs00924547_ml; 4) metabotropic glutamate 5 (GRM5): Hs00168275_ml; 5) beta actin: Hs99999903 ml; and 6) glyceraldehyde β-phosphate dehydrogenase (GAPDH): Hs99999905_ml.

Statistical analysis. All protein measurements for each group were normalized against (β-actin and neuronal specific enolase (NSE) and expressed as ratios of GABRθ/β-actin, GABRρ2/(β-actin, FMRP/β-actin, mGluR5/13-actin, GABRθ/NSE, GABRρ2/NSE, FMRP/NSE, mGluR5/NSE. Statistical analysis was performed as previously described (Fatemi S H, Kneeland R E, Liesch S B, Folsom T D. Fragile X mental retardation protein levels are decreased in major psychiatric disorders [letter]. Schizophr Res 2010a;124: 246-247; Fatemi S H, Folsom T D, Kneeland R E, Liesch S B. Metabotropic glutamate receptor 5 upregulation in children with autism is associated with underexpression of both fragile X mental retardation protein and GABAA receptor beta 3 in adults with autism. Anat Rec 2011a;294:1635-1645; Fatemi S H, King D P, Reutiman T J, Folsom T D, Laurence J A, Lee S, et al. PDE4B polymorphisms and decreased PDE4B expression are associated with schizophrenia. Schizophr Res 2008;101:36-49) with p<0.05 considered significant. Group comparisons were conducted using analysis of variance (ANOVA). Follow-up independent t-tests were then conducted if the results were significant. Group differences on possible confounding factors were explored using chi-square tests for categorical variables and ANOVA for continuous variables. Where group differences were found, analysis of covariance was used to explore these effects on group differences for continuous variables and factorial ANOVA with interaction terms for categorical variables. All analyses were conducted using SPSS v.17 (SPSS Inc, Chicago, Ill.).

Results

Western Blotting Results for GABRθ, GABRρ2, FMRP, and mGluR5 in Lateral Cerebellum

All protein measurements were normalized against β-actin or NSE. In lateral cerebella, Analysis of Variance (ANOVA) identified group differences for GABRθ/β-actin [F(3,48)=5.49, p<0.003], GABRθ/NSE [F(3,48)=5.61, p<0.002], GABRρ2/β-actin [F(3,40)=2.90, p<0.047], GABRρ2/NSE [F(3,40)=3.05, p<0.039], mGluRS monomer/β-actin [F(3,46)=4.15, p<0.011], and mGluR5 monomer/NSE [F(3,46)=5.18, p<0.004] (Table 3). There was a group difference for FMRP/NSE [F(3,50)=4.93, p<0.004] (Table 3).

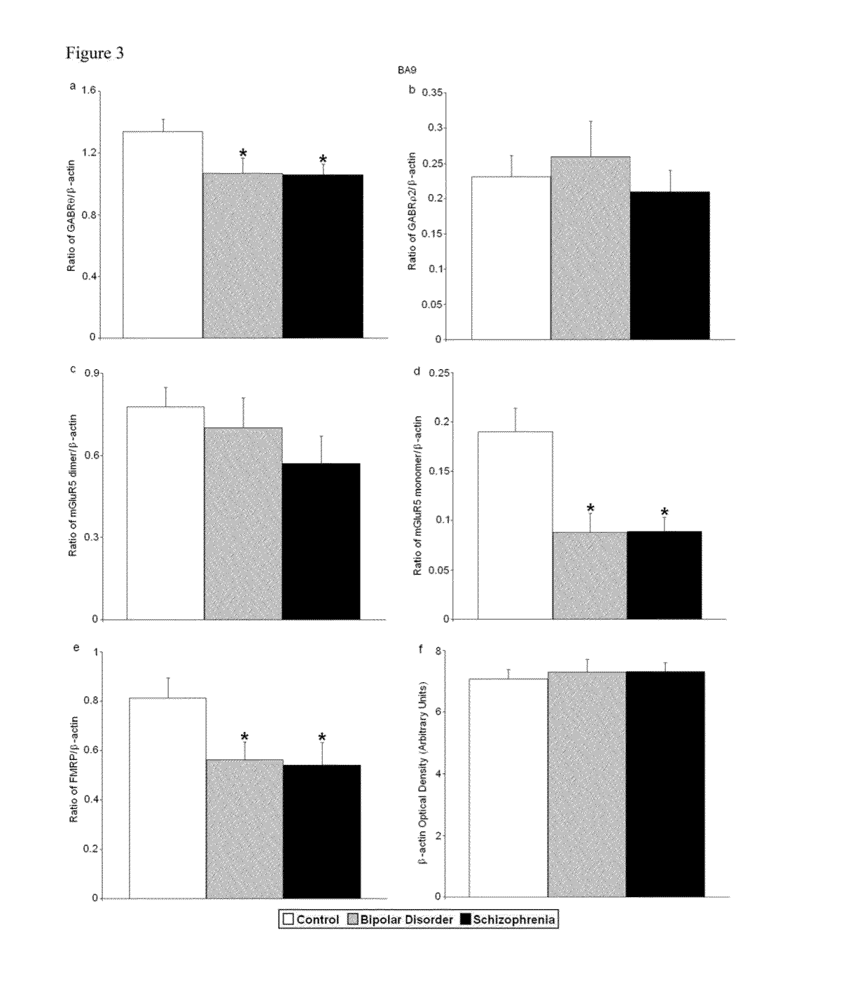

Follow up t-tests found significant reductions in protein for GABRθ/β-actin (p<0.001), GABRθ/NSE (p<0.001), mGluR5 monomer/β-actin (p<0.050), mGluRS monomer/NSE (p<0.030), and FMRP/NSE (p<0.001) in subjects with schizophrenia (Table 3; FIGS. 2 and 3). In lateral cerebella from subjects with bipolar disorder there were significant reductions in GABRθ/β-actin (p<0.012), GABRθ/NSE (p<0.005), mGluR5 monomer/β-actin (p<0.001), mGluR5 monomer/NSE (p<0.004), and FMRP/NSE (p<0.003); and significant increased expression of GABRρ2/β-actin (p<0.0044) and GABRρ2/NSE (p<0.009) (Table 3; FIGS. 2 and 3). In subjects with major depression, follow up t-tests found significant reductions in GABRθ/β-actin (p<0.014), GABRθ/NSE (p<0.012), mGluR5 monomer/NSE (p<0.047), and FMRP/NSE (p<0.001); and significant increased expression of GABRρ2/β-actin (p<0.0085) and GABRρ2/NSE (p<0.006) (Table 3; FIGS. 2 and 3). There were no significant changes in protein levels of mGluR5 dimer in lateral cerebella.

Western Blotting Results for GABRθ, GABRρ2, FMRP, and mGluR5 in BA9

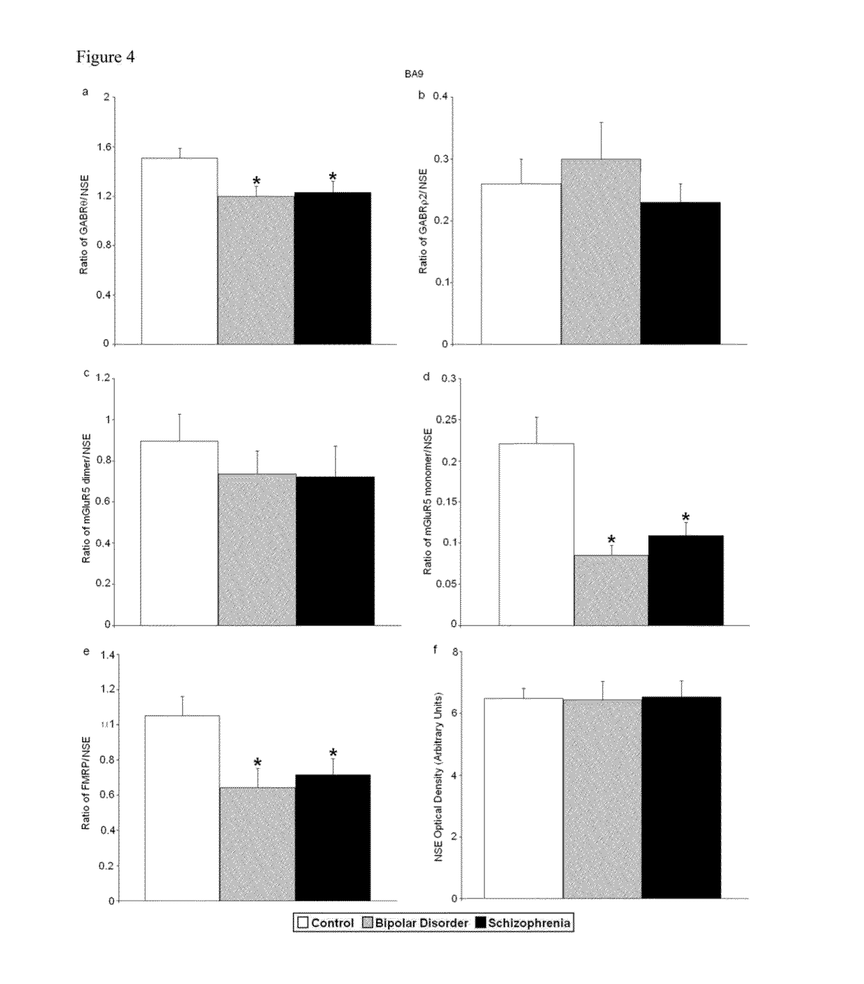

In BA9, ANOVA identified group differences for GABRθ/β-actin [F(2,64)=4.04, p<0.022], GABRθ/NSE [F(2,62)=4.54, p<0.014], mGluR5 monomer/β-actin [F(2,63)=10.72, p<0.001], mGluR5 monomer/NSE [F(2,63)=8.14, p<0.001], FMRP/β-actin [F(2,59)=3.85, p<0.027], and FMRP/NSE [F(2,60)=4.26, p<0.019] (Table 4). Follow up t-tests found significant reductions of GABRθ/β-actin, mGluR5 monomer/β-actin, and FMRP/β-actin (p<0.017, p<0.001, and p<0.018, respectively) and GABRθ/NSE, mGluR5 monomer/NSE, and FMRP/NSE (p<0.019, p<0.003, and p<0.029, respectively) in BA9 of subjects with schizophrenia (Table 4, FIGS. 3 and 4). In subjects with bipolar disorder, follow up t-tests found significant reductions in GABRθ/β-actin, mGluR5 monomer/β-actin, and FMRP/β-actin (p<0.024, p<0.001, and p<0.030, respectively), and GABRθ/NSE, mGluR5 monomer/NSE, and FMRP/NSE (p<0.011, p<0.001, and p<0.011, respectively) (Table 4; FIGS. 3 and 4). There were no significant changes in protein levels for GABRρ2 or mGluR5 dimer in BA9.

Analysis of Confounds for Protein Data in Lateral Cerebellum and BA9

In the analysis of protein data from lateral cerebella, no significant differences were found between groups on hemisphere side, ethnicity, gender, history of substance abuse, severity of alcohol abuse or substance abuse, post mortem interval, age, pH, or brain weight (Table 1). We also compared the groups on family history and suicide and found significant differences (p<0.0001 and p<0.029, respectively) but further analysis revealed that these factors had no significant impact on any of the results. We did find that subjects with schizophrenia and bipolar disorder had significantly longer duration of illness than did those with depression [t(47)=2.47, p<0.018]. Age of onset was significantly later for subjects with major depression compared to subjects with schizophrenia or bipolar disorder [t(41)=3.63, p<0.001]. Analysis of variance controlling for age of onset and duration of illness did find that subjects with depression displayed significantly higher mGluR5dimer/NSE [F(2,31)=4.31, p<0.02) and mGluR5dimer/β-actin [F(2,31)=4.31, p<0.02] than subjects with bipolar disorder or schizophrenia when controlling for duration. However, as mGluR5 dimer values did not change significantly between groups, this finding is not meaningful.

For protein data from BA9, no significant differences were found between diagnostic groups on hemisphere side, history of substance abuse, severity of alcohol abuse or substance abuse, post mortem interval, age, or pH (Table 2). Nor did we find significant differences on use of barbiturates, opiates, amphetamines, cocaine or propoxyphene. We also compared subjects with schizophrenia vs. subjects with bipolar disorder on disease duration, age of onset, and use of antipsychotic, antidepressant, and anticonvulsant medications and found no significant differences. We did find that that 21.1% of bipolar patients died by suicide versus none for the other diagnostic groups [χ²(2)=10.96, p<0.027]. We also found that there were significantly more females [χ²(2)=10.38, p<0.006] in the bipolar group (78.9%) than in either normal controls (41.4%) or subjects with schizophrenia (30%). We also found higher rates of mood stabilizer use in bipolar patients (47.4%) than in schizophrenics (5%), [χ²(1)=9.17, p<0.002]. Further analyses controlling for gender, mood stabilizer use, and suicide found the initial differences on outcomes measures as a function of diagnostic groups to be unchanged.

qRT-PCR Results for GABRθ, GABRρ2, FMRP, and mGluR5 in Lateral Cerebellum and BA9

For qRT-PCR experiments, all values were normalized against both β-actin and GAPDH and these values were averaged. In lateral cerebella, ANOVA identified group differences for GABRQ (GABRθ; p<0.046), GABRR2 (GABRρ2; p<0.017), and GRM5 (mGluR5; p<0.034) (Table 5). There were significantly reduced mRNA values for GABRQ (p<0.016) and GRM5 (p<0.039) in lateral cerebella of subjects with schizophrenia (Table 5), similar to protein changes in the same region. GABRR2 mRNA was significantly increased (p_{<}0.019) in lateral cerebella in subjects with bipolar disorder, mirroring similar changes in protein levels, and GRM5 mRNA expression was significantly reduced (p<0.009) in subjects with major depression (Table 5). In BA9, ANOVA identified group differences for GABRR2 (p<0.003) and GRM5 (p<0.048) (Table 5). In BA9 of subjects with schizophrenia there was significantly increased mRNA for GABRQ (p<0.032). In BA9 of subjects with bipolar disorder there was significantly increased mRNA for GABRR2 (p<0.0001) and significantly reduced mRNA for GRM5 (p<0.041), similar to changes in mGluR5 protein levels in the same region (Table 5). FMR1 mRNA values did not show significant changes in either brain area (Table 5).

Discussion

The current studies demonstrate abnormal processing of mRNA and protein expression for two novel GABA_{A} receptors θ and p2 as well as FMRP and mGluR5 in lateral cerebella and BA9 of subjects with schizophrenia and mood disorders. The most salient results included: 1) FMRP protein levels were significantly decreased in all brain sites in schizophrenia, bipolar disorder, and major depression; 2) mGluR5 protein levels were significantly reduced in all brain sites in schizophrenia and bipolar disorder; 3) mRNA levels for mGluR5 were significantly reduced in lateral cerebellum of subjects with schizophrenia and major depression and BA9 of subjects with bipolar disorder; 4) Protein levels for GABRθ were reduced significantly in all brain sites in schizophrenia, bipolar disorder, and major depression; 5) mRNA levels for GABRθ was elevated significantly in BA9 of subjects with schizophrenia; in contrast mRNA for the same receptor was decreased significantly in lateral cerebellum of subjects with schizophrenia; 6) Protein levels for GABRρ2 were increased significantly in lateral cerebellum of subjects with bipolar disorder and major depression; simultaneously, mRNA for the same receptor was also increased significantly in all brain sites in subjects with bipolar disorder.

The GABRθ gene (GABRQ) is clustered with GABA_{A} receptor epsilon (GABRE) and GABA_{A} receptor alpha 3 (GABRA3) at Xq28 (Korpi E R, Grander G, Lüddens H 2002. Drug interactions at GABA(A) receptors. Prog Neurobiol 2002;67:113-159). In rat, GABRθ mRNA has been shown embryonically (E17/E19) to localize to the hypothalamus, tegmentum, pontine nuclei, and medulla, suggesting a possible role in midbrain development. GABRθ mRNA is expressed in multiple brain regions in human including amygdala, dorsal raphe, hippocampus, hypothalamus, locus coeruleus (LC), and substantia nigra. The LC is relevant to psychiatric disorders as it is the largest noradrenergic nucleus and plays important roles in regulation of anxiety states, vigilance, attention and memory functions. GABRθ forms a functional receptor when co-expressed with alpha, beta, and gamma subunits (Bonnert T P, McKernan R M, Farrar S, le Bourdellès B, Heavens R P, Smith D W, et al. Theta, a novel gamma-aminobutyric acid type A receptor subunit. Proc Natl Acad Sci USA 1999;96: 9891-9896). The functional properties of GABA_{A} receptors that include the θ subunit have not been well characterized (Pape J R, Bertrand S S, Lafon P, Odessa M F, Chaigniau M, Stiles J K, et al. Expression of GABA(A) receptor alpha3-, theta-, and epsilon-subunit mRNAs during rat CNS development and immunolocalization of the epsilon subunit in developing postnatal spinal cord. Neuroscience 2009;160:85-96).

Recent studies have investigated possible associations of the gene that codes GABRθ (GABRQ) with multiple disorders (Breuer R, Hamshere M L, Strohmaier J, Mattheisen M, Degenhardt F, Meier S, et al. Independent evidence for the selective influence of GABA(A) receptors on one component of the bipolar disorder phenotype. Mol Psychiatry 2011;16:587-589; Craddock N, Jones L, Jones I R, Kirov G, Green E K, Grozeva D, et al. Strong genetic evidence for a selective influence of GABAA receptors on a component of the bipolar disorder phenotype. Mol Psychiatry 2010;15:146-153; Fernandez F, Esposito T, Lea R A, Colson N J, Ciccodicola A, Gianfrancesco F, Griffiths L R. Investigation of gamma-aminobutyric acid (GABA) A receptors genes and migraine susceptibility. BMC Med Genet 2008;9:109; Garcia-Martin, E., Martinez, C., Alonso-Navarro, H., Benito-Leon, J., Lorenzo-Betancor, O. Pastor P, et al. Gamma-aminobutyric acid GABRA4, GABRE, and GABRQ receptor polymorphisms and risk for essential tremor. Pharmacogenet Genomics 2011;21:436-439). However, single nucleotide polymorphisms (SNPs) of GABRQ were not associated with susceptibility to bipolar disorder, migraine, or essential tremor. However, the GABRQ-478F allele showed an association with improvement of tremor with ethanol use among men.

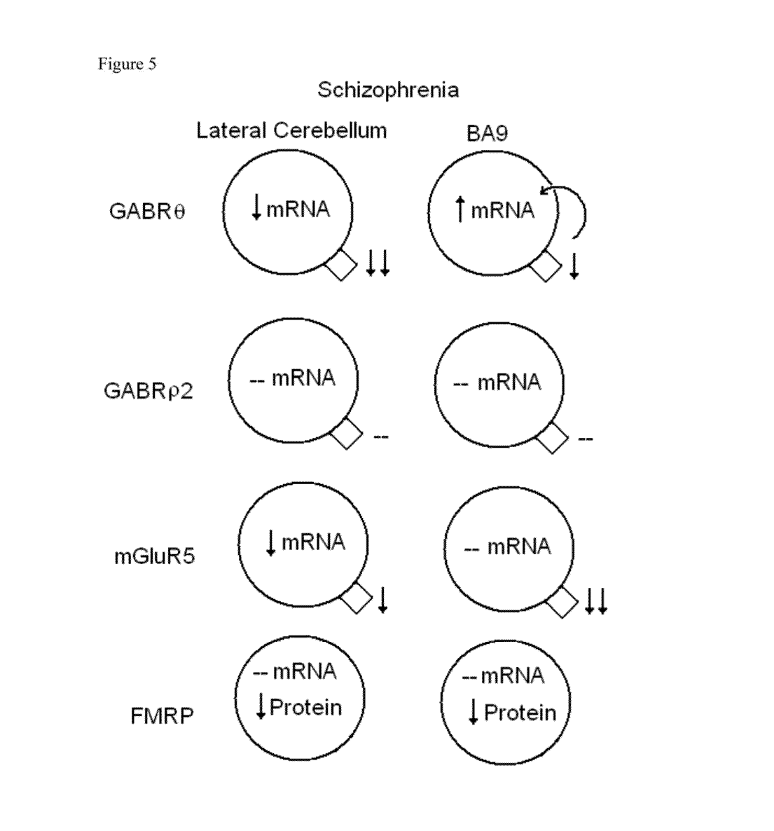

The levels of GABRθ receptor protein are reduced significantly in both BA9 and lateral cerebellum of subjects with schizophrenia (FIG. 5). In contrast, mRNA for GABRθ receptor is downregulated in lateral cerebella while in BA9 its mRNA is upregulated (FIG. 5). As both mRNA and protein are concordantly downregulated in lateral cerebellum, a severe chronic receptor deficit may be responsible for our observed results; while in BA9, increased mRNA expression may be a compensatory response to chronic receptor downregulation, suggesting that different mechanisms may be at work (FIG. 5).

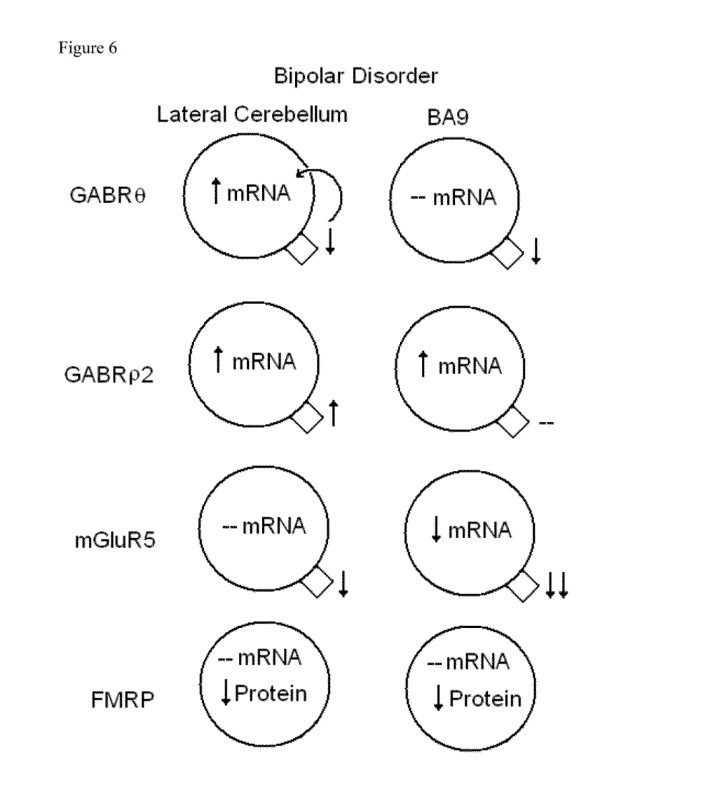

In subjects with bipolar disorder, GABRθ receptor protein is reduced in lateral cerebellum while its mRNA is upregulated (FIG. 6). By the same token, protein for this receptor is downregulated in BA9 with its mRNA level unchanged (FIG. 6). Here, the mechanisms for these alterations may again be different in the two brain sites. In lateral cerebellum, chronic GABRθ protein downregulation could lead to upregulation of its mRNA in a feedback loop. In BA9, normal mRNA levels with decreased protein levels indicate a defective step either in processing of protein in rough endoplasmic reticulum (rER) or subsequent cell compartments (such as golgi or secretory vesicles) leading to reduced protein production (FIG. 6).

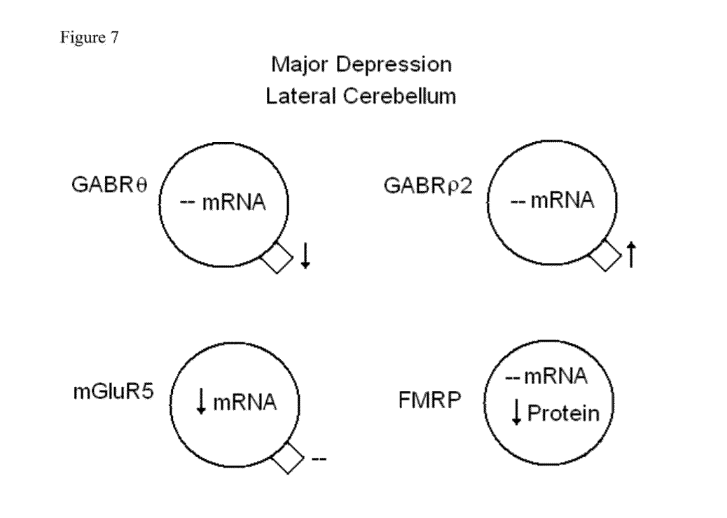

In subjects with major depression, while protein levels for GABRθ are reduced significantly in lateral cerebellum, its mRNA levels remain normal (FIG. 7). This scenario again indicates that the deficit lies at rER or a subsequent cellular compartment causing the chronic receptor protein downregulation (FIG. 7). In the absence of available BA9 tissue, the fate of GABRθ in major depression will await future determinations.

The gene that codes GABRρ2 (GABRR2) is localized to 6q15 (Ma DQ, Whitehead P L, Menold M M, Martin E R, Ashley-Koch A E, Mei H, et al. Identification of significant association and gene-gene interaction of GABA receptor subunit genes in autism. Am J Hum Genet 2005;77:377-388), GABRρ2 mRNA is widely distributed in brain including prefrontal cortex, hippocampus, and cerebellum. In adult rat cerebellum, GABRρ2 has been localized to Purkinje cells and basket-like cells only. GABRρ2 has been shown to associate with alphal (α1) and gamma2 (γ2) receptor subunits (Milligan C J, Buckley N J, Garret M, Deuchars J, Deuchars S A. Evidence for inhibition mediated by coassembly of GABAA and GABAC receptor subunits in native central neurons. J Neurosci 2004;24:7241-7250). In cerebellum, GABA_{A} receptors that include the ρ2 subunit help mediate a component of phasic inhibitory GABAergic transmission at intemeuron-Purkinje cell synapses.

A recent study has demonstrated an association between an SNP of GABRR2 (GABRρ2) (rs1570932) and a component of the bipolar phenotype, namely psychotic symptoms, similar to those experienced by subjects with schizophrenia (Breuer R, Hamshere M L, Strohmaier J, Mattheisen M, Degenhardt F, Meier S, et al. Independent evidence for the selective influence of GABA(A) receptors on one component of the bipolar disorder phenotype. Mol Psychiatry 2011;16:587-589). A second study found an SNP (rs12201676) associated with bipolar disorder that is flanked by GABRRI (15 kb away) and GABRR2 (17 kb away) genes (Wang K S, Liu X F, Aragam N. A genome-wide meta-analysis identifies novel loci associated with schizophrenia and bipolar disorder. Schizophr Res 2010;124:192-199). GABRR2 has also been associated with alcoholism.

Levels of GABRρ2 mRNA and protein did not change in lateral cerebellum or BA9 of subjects with schizophrenia (FIG. 5). However, in subjects with bipolar disorder, a concordant and significant increase was observed in mRNA and protein levels of GABRρ2 in lateral cerebellum indicating chronic upregulation in gene and protein product in this disorder (FIG. 6). Interestingly, in BA9 of bipolar subjects, GABRρ2 mRNA levels were also elevated significantly but no protein change was observed (FIG. 6). In major depression, GABRρ2 protein levels were also elevated but without any change in mRNA indicating abnormalities in processing GABRρ2 protein in rER compartment or subsequent cellular stations (FIG. 7). Thus, GABRρ2 changes were confined to brains of subjects with mood disorders and not seen in schizophrenia and, at least in the case of bipolar disorder, reflect upregulation of GABRρ2 mRNA/protein.

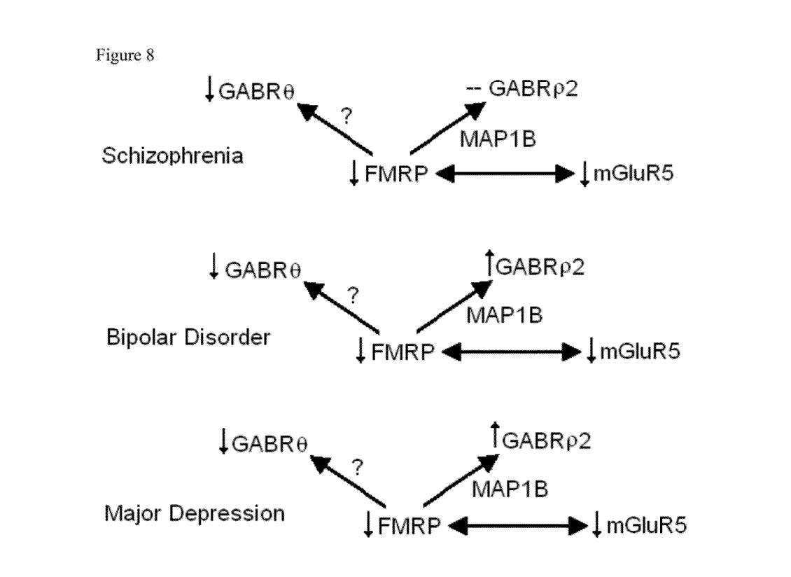

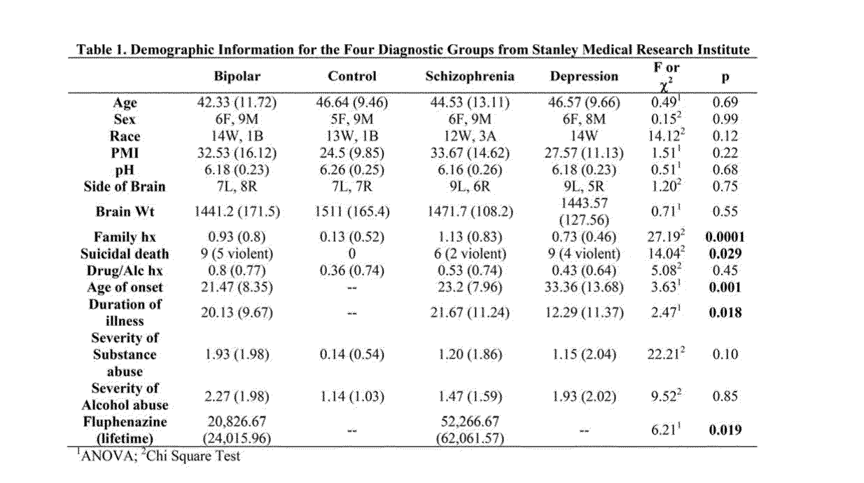

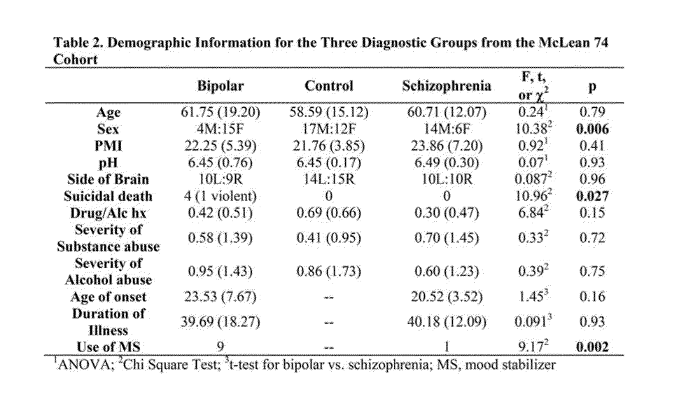

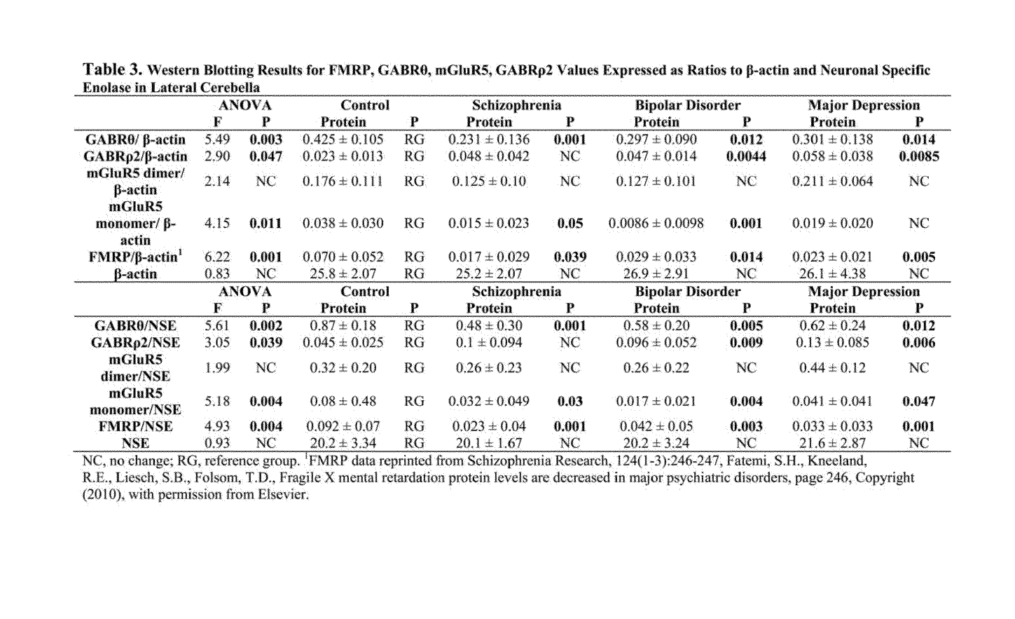

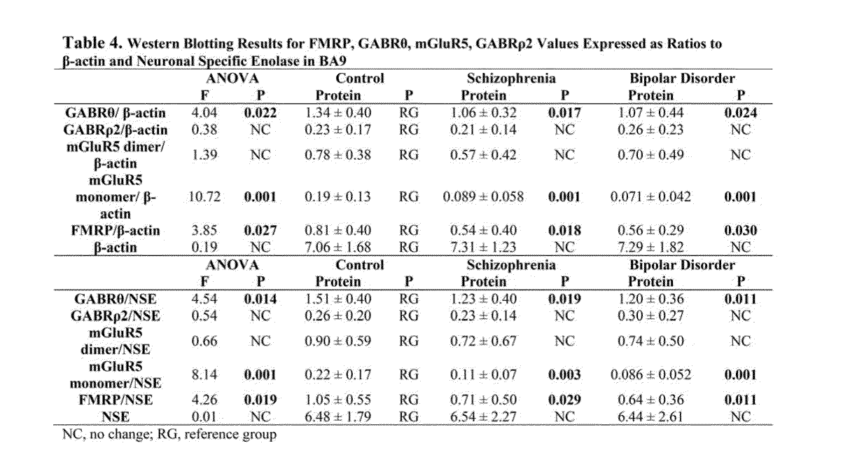

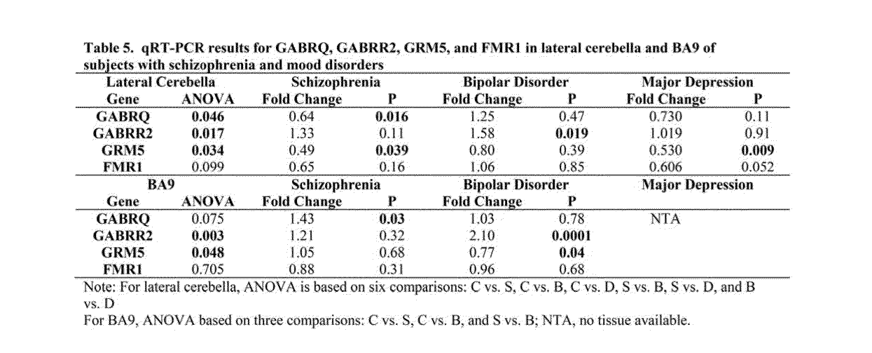

The FMR1 gene is located at Xq27.3. FMRP has been shown to localize in multiple regions of neurons including the soma, dendrites, synaptic spines, and the axon. FMRP controls multiple post-transcriptional events including splicing, nuclear export, and translation. FMRP protein is significantly downregulated in schizophrenia, bipolar disorder, and major depression in lateral cerebellum and in BA9 for subjects with schizophrenia and bipolar disorder in the absence of any mRNA abnormalities (FIGS. 5-7). This picture is similar to what we have described in idiopathic cases of autism without evidence of any effects in gene for FMRP and thus replicative of posttranscriptional abnormalities affecting protein synthesis (FIG. 8). Changes in mRNA expression do not always correlate with similar changes in protein expression, including expression of FMRP. A recent study found that in subjects with the FMR1 premutation (expanded 5′ CGG repeat but without full symptoms of FXS), there was both significantly increased FMR1 mRNA and significantly reduced levels of FMRP. Similarly, the recent findings of Kovacs et al. demonstrated downregulation of FMRP protein levels in the absence of any change in FMR1 mRNA or expansion of the 5′ CGG triplet repeat in peripheral blood lymphocytes of subjects with schizophrenia. As reduced FMRP expression has been identified in four major psychiatric disorders, identifying the posttranscriptional abnormalities that may be responsible for this reduction would have a major impact on the etiology and treatment of these disorders. Additionally, verification of reduced FMRP protein levels in peripheral blood lymphocytes of subjects with schizophrenia confirm our data at least in schizophrenia and validate our additional new findings of reduced FMRP in BA9 of subjects with schizophrenia.

The gene for mGluR5 is located at 11q14.2-q14.3. mGluR5, like other metabotropic glutamate receptors contain seven membrane-spanning domains and a large extracellular N-terminus and are G-protein coupled. Metabotropic glutamate receptors are found throughout the CNS with high concentrations in cerebral cortex, hippocampus, striatum, hypothalamus, midbrain, cerebellum, medulla, and pons. There were concordant and significant reductions in levels of mRNA and protein for mGluRS in lateral cerebellum of subjects with schizophrenia (FIG. 5). In BA9, despite significant reductions in protein levels, mRNA levels were normal (FIG. 5). Thus, protein levels for mGluRS were downregulated in both brain sites in schizophrenia. In a similar vein, mGluR5 protein levels were reduced significantly in both lateral cerebellum and BA9 in subjects with bipolar disorder despite normal mRNA levels indicating posttranscriptional abnormalities in the pathway for mGluR5 protein synthesis (FIG. 6). Interestingly, in lateral cerebellum of subjects with major depression, despite downregulated mRNA for mGluR5, the protein levels were normal (FIG. 7). It is possible that unknown mechanisms affecting transit for protein rescue the product for release despite low turnover for its mRNA, in major depression.

Recently, Matosin et al. (Matosin N, Frank E, Deng C, Huang X-F, Newell K A. Metabotropic glutamate receptor 5 binding and protein expression in schizophrenia and following antipsychotic drug treatment. Schizophr Res 2013 (in press)) showed no significant alteration in mGluR5 binding density or mGluR5 protein levels in dorsolateral prefrontal cortex of subjects with schizophrenia. However, close inspection of their Western blotting data showed highly oversaturated bands for the monomeric mGluR5 protein levels for both control and subjects with schizophrenia, potentially masking any differences between the two groups. While several others did not show any change in mGluR5 protein or mRNA in PFC of subjects with schizophrenia, these results could be due to use of different analytic techniques or brain regional effects. However, others have reported decreases in mGluR5 mRNA in PFC of subjects with schizoaffective disorder (Volk D W, Eggan S M, Lewis D A. Alterations in metabotropic glutamate receptor 1α and regulator of G protein signaling 4 in the prefrontal cortex in schizophrenia. Am JPsychiatry 2010;167:1489-1498), and in those with major depression (Deschwanden A, Karolewicz B, Feyissa A M, Treyer V, Ametamey S M, Johayem A, et al. Reduced metabotropic glutamate receptor 5 density in major depression determined by [(11)C]ABP688 PET and postmortem study. Am J Psychiatry 2011;168:724-734), supporting our current data showing significant decreases in mGluR5 protein levels in lateral cerebellum and BA9 of subjects with schizophrenia and bipolar disorder and decreases in mRNA levels in lateral cerebella of subjects with schizophrenia and major depression and BA9 of subjects with bipolar disorder. Additionally, we have previously observed increased expression of mGluR5 protein in BA9 and cerebellar vermis of children with autism (FIG. 8). However, while there is a great deal of overlap in the symptomologies of autism and FXS, there is less so between FXS and schizophrenia and mood disorders.

Interactions between the aforementioned four proteins may alter GABAergic transmission. The cytoplasmic domains of GABRρ1 and GABRρ2 interact with MAP1B (FIG. 8). Disruption of ρ-MAP1B interactions leads to a doubling of the inward current of GABAt_{c} receptors from bipolar cells in retinal slices in the presence of low levels of GABA. MAP1B mRNA is targeted by FMRP

(FIG. 8) and in FMR1 KO mice there is an abnormal upregulation of MAP1B. With reduced expression of FMRP one might speculate that there would be increased expression of MAP1B in subjects with schizophrenia and mood disorders. However, a preliminary study involving anterior cingulate cortex found reduced expression of MAP1B protein in subjects with bipolar disorder, with no change in subjects with schizophrenia or major depression. (Bouras C, Kövari E, Hof P R, Riederer B M, Giannakopoulos P. Anterior cingulate cortex pathology in schizophrenia and bipolar disorder. Acta Neuropathol 2001;102: 373-379) Altered expression of MAP1B could in turn cause changes in GABAergic neurotransmission through GABA receptors that contain ρ subunits.

In conclusion, FMRP is significantly downregulated in lateral cerebella and BA9 from subjects with schizophrenia, bipolar disorder, and major depression potentially causing GABA receptor changes and altered expression of mGluR5 in the three disorders in the absence of any FMR1 chromosomal abnormalities. Additionally, we have identified selective abnormalities in mRNA and protein levels of two novel GABA_{A} receptors, namely GABRθ and GABRρ2 in subjects with schizophrenia and mood disorders. These changes could potentially explain changes in GABAergic transmission and consequent deficits associated with these disorders including anxiety, panic, and impaired learning and memory.

### Example 2

### mRNA and Protein Expression for Novel GABA_{A} Receptors Θ And P2 are Altered in Autism

Gamma-aminobutyric acid (GABA) is the main inhibitory neurotransmitter in the brain. GABAergic receptor abnormalities have been documented in several major psychiatric disorders including schizophrenia, mood disorders, and autism. Abnormal expression of mRNA and protein for multiple GABA receptors has also been observed in multiple brain regions leading to alterations in the balance between excitatory/inhibitory signaling in the brain with potential profound consequences for normal cognition and maintenance of mood and perception. Altered expression of GABA_{A} receptor subunits has been documented in fragile X mental retardation 1 (FMR1) knockout mice, suggesting that loss of its protein product, fragile X mental retardation protein (FMRP), impacts GABA_{A} subunit expression. Recent postmortem studies have shown reduced expression of FMRP in the brains of subjects with schizophrenia, bipolar disorder, major depression, and autism. FMRP acts as a translational repressor and, under normal conditions, inhibits metabotropic glutamate receptor 5 (mGluR5)-mediated signaling. In fragile X syndrome (FXS), the absence of FMRP is hypothesized to lead to unregulated mGluR5 signaling, ultimately resulting in the behavioral and intellectual impairments associated with this disorder. Changes have been identified in mGluR5 expression in autism, schizophrenia, and mood disorders. Fatemi S H and Folsom T D, GABA receptor subunit distribution and FMRP—mGluR5 signaling abnormalities in the cerebellum of subjects with schizophrenia, mood disorders, and autism, Schizophr Res. 2014 Nov. 25. pii: S0920-9964(14)00549-0 (incorporated by reference herein).

Protein and mRNA levels were measured for nine gamma-aminobutyric acid A (GABAA) receptor subunits in three brain regions (cerebellum, superior frontal cortex, and parietal cortex) in subjects with autism versus matched controls. Changes in mRNA were observed for a number of GABA_{A} and GABA_{B} subunits and overall reduced protein expression for GABA_{A} receptor alpha 6 (GABRα6), GABA_{A} receptor beta 2 (GABRβ2), GABA_{A} receptor delta (GABRδ), GABA_{A} receptor epsilon (GABRε), GABA_{A} receptor gamma 2 (GABRγ2), GABA_{A} receptor theta (GABRθ), and GABA_{A} receptor rho 2 (GABRρ2) in superior frontal cortex from subjects with autism. THE data demonstrate systematic changes in GABA_{A&B} subunit expression in brains of subjects with autism, which may help explain the presence of cognitive abnormalities in subjects with autism. Fatemi HfS et al., Downregulation of GABAA Receptor Protein Subunits α6, β2, δ, ε, γ2, and ρ2 in Superior Frontal Cortex of Subjects with Autism, J Autism Dev Disord (2014) 44:1833-1845 (incorporated by reference herein).

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

TABLE 1
Demographic Information for the Four Diagnostic
Groups from Stanley Medical Research Institute

| | Bipolar | Control | Schizophrenia | Depression | F or χ² | p |
|---|---|---|---|---|---|---|
| Age | 42.33 | 46.64 | 44.53 | 46.57 | 0.49¹ | 0.69 |
| | (11.72) | (9.46) | (13.11) | (9.66) | | |
| Sex | 6F, 9M | 5F, 9M | 6F, 9M | 6F, 8M | 0.15² | 0.99 |
| Race | 14W, 1B | 13W, 1B | 12W, 3A | 14W | 14.12² | 0.12 |
| PMI | 32.53 | 24.5 | 33.67 | 27.57 | 1.51¹ | 0.22 |
| | (16.12) | (9.85) | (14.62) | (11.13) | | |
| pH | 6.18 | 6.26 | 6.16 | 6.18 | 0.51¹ | 0.68 |
| | (0.23) | (0.25) | (0.26) | (0.23) | | |
| Side of Brain | 7L, 8R | 7L, 7R | 9L, 6R | 9L, 5R | 1.20² | 0.75 |
| Brain Wt | 1441.2 | 1511 | 1471.7 | 1443.57 | 0.71¹ | 0.55 |
| | (171.5) | (165.4) | (108.2) | (127.56) | | |
| Family hx | 0.93 | 0.13 | 1.13 | 0.73 | 27.19² | 0.0001 |
| | (0.8) | (0.52) | (0.83) | (0.46) | | |
| Suicidal death | 9 (5 violent) | 0 | 6 (2 violent) | 9 (4 violent) | 14.04² | 0.029 |
| Drug/Alc hx | 0.8 | 0.36 | 0.53 | 0.43 | 5.08² | 0.45 |
| | (0.77) | (0.74) | (0.74) | (0.64) | | |
| Age of onset | 21.47 | — | 23.2 | 33.36 | 3.63¹ | 0.001 |
| | (8.35) | | (7.96) | (13.68) | | |
| Duration of | 20.13 | — | 21.67 | 12.29 | 2.47¹ | 0.018 |
| illness | (9.67) | | (11.24) | (11.37) | | |
| Severity of | 1.93 | 0.14 | 1.20 | 1.15 | 22.21² | 0.10 |
| Substance | (1.98) | (0.54) | (1.86) | (2.04) | | |
| abuse | | | | | | |
| Severity of | 2.27 | 1.14 | 1.47 | 1.93 | 9.52² | 0.85 |
| Alcohol abuse | (1.98) | (1.03) | (1.59) | (2.02) | | |
| Fluphenazine | 20,826.67 | — | 52,266.67 | — | 6.21¹ | 0.019 |
| (lifetime) | (24,015.96) | | (62,061.57) | | | |

¹ANOVA;
²Chi Square Test

TABLE 2
Demographic Information for the Three Diagnostic Groups from the
McLean 74 Cohort

| | | | | F, t, | |
|---|---|---|---|---|---|
| | Bipolar | Control | Schizophrenia | or χ² | p |
| Age | 61.75 (19.20) | 58.59 (15.12) | 60.71 (12.07) | 0.24¹ | 0.79 |
| Sex | 4M:15F | 17M:12F | 14M:6F | 10.38² | 0.006 |
| PMI | 22.25 (5.39) | 21.76 (3.85) | 23.86 (7.20) | 0.92¹ | 0.41 |
| pH | 6.45 (0.76) | 6.45 (0.17) | 6.49 (0.30) | 0.07¹ | 0.93 |
| Side of Brain | 10L:9R | 14L:15R | 10L:10R | 0.087² | 0.96 |
| Suicidal death | 4 (1 violent) | 0 | 0 | 10.96² | 0.027 |
| Drug/Alc hx | 0.42 (0.51) | 0.69 (0.66) | 0.30 (0.47) | 6.84² | 0.15 |
| Severity of | 0.58 (1.39) | 0.41 (0.95) | 0.70 (1.45) | 0.33² | 0.72 |
| Substance abuse | | | | | |
| Severity of | 0.95 (1.43) | 0.86 (1.73) | 0.60 (1.23) | 0.39² | 0.75 |
| Alcohol abuse | | | | | |
| Age of onset | 23.53 (7.67) | — | 20.52 (3.52) | 1.45³ | 0.16 |
| Duration of Illness | 39.69 (18.27) | — | 40.18 (12.09) | 0.091³ | 0.93 |
| Use of MS | 9 | — | 1 | 9.17² | 0.002 |

¹ANOVA; ²Chi Square Test; ³t-test for bipolar vs. schizophrenia; MS, mood stabilizer

TABLE 3
Western Blotting Results for FMRP, GABRθ, mGluR5, GABRρ2 Values Expressed as
Ratios to β-actin and Neuronal Specific Enolase in Lateral Cerebella

| | ANOVA | | Control | | Schizophrenia | | Bipolar Disorder | | Major Depression | |
|---|---|---|---|---|---|---|---|---|---|---|
| | F | P | Protein | P | Protein | P | Protein | P | Protein | P |
| GABRθ/ β-actin | 5.49 | 0.003 | 0.425 ± 0.105 | RG | 0.231 ± 0.136 | 0.001 | 0.297 ± 0.090 | 0.012 | 0.301 ± 0.138 | 0.014 |
| GABRρ2/β-actin | 2.90 | 0.047 | 0.023 ± 0.013 | RG | 0.048 ± 0.042 | NC | 0.047 ± 0.014 | 0.0044 | 0.058 ± 0.038 | 0.0085 |
| mGluR5 dimer/ | 2.14 | NC | 0.176 ± 0.111 | RG | 0.125 ± 0.10 | NC | 0.127 ± 0.101 | NC | 0.211 ± 0.064 | NC |
| β-actin | | | | | | | | | | |
| mGluR5 | 4.15 | 0.011 | 0.038 ± 0.030 | RG | 0.015 ± 0.023 | 0.05 | 0.0086 ± 0.0098 | 0.001 | 0.019 ± 0.020 | NC |
| monomer/β-actin | | | | | | | | | | |
| FMRP/β-actin¹ | 6.22 | 0.001 | 0.070 ± 0.052 | RG | 0.017 ± 0.029 | 0.039 | 0.029 ± 0.033 | 0.014 | 0.023 ± 0.021 | 0.005 |
| β-actin | 0.83 | NC | 25.8 ± 2.07 | RG | 25.2 ± 2.07 | NC | 26.9 ± 2.91 | NC | 26.1 ± 4.38 | NC |
| GABRθ/NSE | 5.61 | 0.002 | 0.87 ± 0.18 | RG | 0.48 ± 0.30 | 0.001 | 0.58 ± 0.20 | 0.005 | 0.62 ± 0.24 | 0.012 |
| GABRρ2/NSE | 3.05 | 0.039 | 0.045 ± 0.025 | RG | 0.1 ± 0.094 | NC | 0.096 ± 0.052 | 0.009 | 0.13 ± 0.085 | 0.006 |
| mGluR5 | 1.99 | NC | 0.32 ± 0.20 | RG | 0.26 ± 0.23 | NC | 0.26 ± 0.22 | NC | 0.44 ± 0.12 | NC |
| dimer/NSE | | | | | | | | | | |
| mGluR5 | 5.18 | 0.004 | 0.08 ± 0.48 | RG | 0.032 ± 0.049 | 0.03 | 0.017 ± 0.021 | 0.004 | 0.041 ± 0.141 | 0.047 |
| monomer/NSE | | | | | | | | | | |
| FMRP/NSE | 4.93 | 0.004 | 0.092 ± 0.07 | RG | 0.023 ± 0.04 | 0.001 | 0.042 ± 0.05 | 0.003 | 0.033 ± 0.033 | 0.001 |
| NSE | 0.93 | NC | 20.2 ± 3.34 | RG | 20.1 ± 1.67 | NC | 20.2 ± 3.24 | NC | 21.6 ± 2.87 | NC |

NC, no change;
RG, reference group.
¹FMRP data reprinted from Schizophrenia Research, 124(1-3): 246-247, Fatemi, S.H. , Kneeland, R.E., Liesch, S.B. , Folsom, T.D. , Fragile X mental retardation protein levels are decreased in major psychiatric disorders, page 246, Copyright (2010), with permission from Elsevier.

TABLE 4
Western Blotting Results for FMRP, GABRθ, mGluR5, GABRρ2 Values Expressed
as Ratios to β-actin and Neuronal Specific Enolase in BA9

| | ANOVA | | Control | | Schizophrenia | | Bipolar Disorder | |
|---|---|---|---|---|---|---|---|---|
| | F | P | Protein | P | Protein | P | Protein | P |
| GABRθ/β-actin | 4.04 | 0.022 | 1.34 ± 0.40 | RG | 1.06 ± 0.32 | 0.017 | 1.07 ± 0.44 | 0.024 |
| GABRρ2/β-actin | 0.38 | NC | 0.23 ± 0.17 | RG | 0.21 ± 0.14 | NC | 0.26 ± 0.23 | NC |
| mGluR5 dimer/ | 1.39 | NC | 0.78 ± 0.38 | RG | 0.57 ± 0.42 | NC | 0.70 ± 0.49 | NC |
| β-actin | | | | | | | | |
| mGluR5 | 10.72 | 0.001 | 0.19 ± 0.13 | RG | 0.089 ± 0.058 | 0.001 | 0.071 ± 0.042 | 0.001 |
| monomer/β- | | | | | | | | |
| actin | | | | | | | | |
| FMRP/β-actin | 3.85 | 0.027 | 0.81 ± 0.40 | RG | 0.54 ± 0.40 | 0.018 | 0.56 ± 0.29 | 0.030 |
| β-actin | 0.19 | NC | 7.06 ± 1.68 | RG | 7.31 ± 1.23 | NC | 7.29 ± 1.82 | NC |
| GABRθ/NSE | 4.54 | 0.014 | 1.51 ± 0.40 | RG | 1.23 ± 0.40 | 0.019 | 1.20 ± 0.36 | 0.011 |
| GABRρ2/NSE | 0.54 | NC | 0.26 ± 0.20 | RG | 0.23 ± 0.14 | NC | 0.30 ± 0.27 | NC |
| mGluR5 | 0.66 | NC | 0.90 ± 0.59 | RG | 0.72 ± 0.67 | NC | 0.74 ± 0.50 | NC |
| dimer/NSE | | | | | | | | |
| mGluR5 | 8.14 | 0.001 | 0.22 ± 0.17 | RG | 0.11 ± 0.07 | 0.003 | 0.086 ± 0.052 | 0.001 |
| monomer/NSE | | | | | | | | |
| FMRP/NSE | 4.26 | 0.019 | 1.05 ± 0.55 | RG | 0.71 ± 0.50 | 0.029 | 0.64 ± 0.36 | 0.011 |
| NSE | 0.01 | NC | 6.48 ± 1.79 | RG | 6.54 ± 2.27 | NC | 6.44 ± 2.61 | NC |

NC, no change;
RG, reference group

TABLE 5
qRT-PCR results for GABRQ, GABRR2, GRM5, and FMRI in lateral cerebella
and BA9 of subjects with schizophrenia and mood disorders

| Lateral Cerebella | | Schizophrenia | | Bipolar Disorder | | Major Depression | |
|---|---|---|---|---|---|---|---|
| Gene | ANOVA | Fold Change | P | Fold Change | P | Fold Change | P |
| GABRQ | 0.046 | 0.64 | 0.016 | 1.25 | 0.47 | 0.730 | 0.11 |
| GABRR2 | 0.017 | 1.33 | 0.11 | 1.58 | 0.019 | 1.019 | 0.91 |
| GRM5 | 0.034 | 0.49 | 0.039 | 0.80 | 0.39 | 0.530 | 0.009 |
| FMR1 | 0.099 | 0.65 | 0.16 | 1.06 | 0.85 | 0.606 | 0.052 |
| BA9 | | Schizophrenia | | Bipolar Disorder | | | |
| Gene | ANOVA | Fold Change | P | Fold Change | P | Major Depression | |
| GABRQ | 0.075 | 1.43 | 0.03 | 1.03 | 0.78 | NTA | |
| GABRR2 | 0.003 | 1.21 | 0.32 | 2.10 | 0.0001 | | |
| GRM5 | 0.048 | 1.05 | 0.68 | 0.77 | 0.04 | | |
| FMR1 | 0.705 | 0.88 | 0.31 | 0.96 | 0.68 | | |

Note:
For lateral cerebella, ANOVA is based on six comparisons: C vs. S, C vs. B, C vs. D, S vs. B, S vs. D, and B vs. D
For BA9, ANOVA based on three comparisons: C vs. S, C vs. B, and S vs. B;
NTA, no tissue available.

What is claimed is:

1. A method of determining the increased likelihood of having or developing schizophrenia in a patient, comprising
(a) obtaining a physiological sample from the patient;
(b) quantifying levels of FMRP protein, mGluR5 protein, and GABRθ protein in the sample, and comparing the levels to a control protein sample, and/or quantifying levels of mGluR5 mRNA and GABRθ mRNA in the sample, and comparing the levels to a control mRNA sample; and
(c) determining that the patient has schizophrenia or an increased likelihood of developing schizophrenia (i) based upon the level of FMRP protein, mGluR5 protein, and GABRθ protein in the sample, wherein a lower level of FMRP protein, mGluR5 protein, and GABRθ protein in the sample as compared to the control indicates that the patient has schizophrenia or has an increased likelihood of developing schizophrenia, (ii) based upon the level of mGluR5 mRNA and GABRθ mRNA in the sample, wherein a lower level of mGluR5 mRNA and GABRθ mRNA in the sample as compared to the control indicates that the patient has schizophrenia or has an increased likelihood of developing schizophrenia, or (iii) based upon the level of FMRP protein, mGluR5 protein, and GABRθ protein in the sample, and based upon the level of mGluR5 mRNA and GABRθ mRNA in the sample, wherein a lower level of FMRP protein, mGluR5 protein, and GABRθ protein in the sample and a lower level of mGluR5 mRNA and GABRθ mRNA in the sample as compared to the control indicates that the patient has schizophrenia or has an increased likelihood of developing schizophrenia.

2. The method of claim 1, wherein the physiological sample is brain tissue, blood or blood product, or cerebral spinal fluid (CSF).

3. The method of claim 1, wherein the protein level is determined by means of a Western blot.

4. The method of claim 1, wherein the mRNA level is determined by means of quantitative real-time polymerase chain reaction (qRT-PCR).

5. A method of determining the increased likelihood of having or developing bipolar disorder in a patient, comprising
(a) obtaining a physiological sample from the patient;
(b) quantifying levels of FMRP protein, mGluR5 protein, GABRθ protein, and GABRρ2 protein in the sample, and comparing the levels to a control sample; and
(c) determining that the patient has bipolar disorder or an increased likelihood of developing bipolar disorder based upon the level of FMRP protein, mGluR5 protein, GABRθ protein, and GABRρ2 protein in the sample, wherein a lower level of FMRP protein, mGluR5 protein, and GABRθ protein, and a higher level of GABRρ2 protein in the sample as compared to the control indicates that the patient has bipolar disorder or has an increased likelihood of developing bipolar disorder.

6. The method of claim 5, wherein the physiological sample is brain tissue, blood or cerebral spinal fluid (CSF).

7. The method of claim 5, wherein the protein level is determined by means of a Western blot.

8. The method of claim 5, further comprising at step (b) quantifying levels of GABroh2 mRNA in the sample, and comparing the levels to a control sample, wherein at step (c) a lower level of FMRP protein, mGluR5 protein, and GABRθ protein, and a higher level of GABRρ2 protein and GABroh2 mRNA in the sample as compared to the control indicates that the patient has bipolar disorder or has an increased likelihood of developing bipolar disorder.

9. The method of claim 8, wherein the mRNA level is determined by means of quantitative real-time polymerase chain reaction (qRT-PCR).

10. A method of determining the increased likelihood of having or developing major depressive disorder (MDD) in a patient, comprising
(a) obtaining a physiological sample from the patient;
(b) quantifying levels of FMRP protein, GABRθ protein and GABRρ2 protein in the sample, and comparing the levels to a control sample; and
(c) determining that the patient has MDD or an increased likelihood of developing MDD based upon the level of FMRP protein, GABRθ protein, and GABRρ2 protein in the sample, wherein a lower level of FMRP protein and GABRθ protein, and a higher level of GABRρ2 protein in the sample as compared to the control indicates that the patient has MDD or has an increased likelihood of developing MDD.

11. The method of claim 10, wherein the physiological sample is brain tissue, blood or blood product, or cerebral spinal fluid (CSF).

12. The method of claim 10, wherein the protein level is determined by means of a Western blot.

13. The method of claim 10, further comprising at step (b) quantifying levels of mGluR5 mRNA in the sample, and comparing the levels to a control sample, wherein at step (c) a lower level of FMRP protein, GABRθ protein and mGluR5 mRNA, and a higher level of GABRρ2 protein in the sample as compared to the control indicates that the patient has MDD or has an increased likelihood of developing MDD.

14. The method of claim 13, wherein the mRNA level is determined by means of quantitative real-time polymerase chain reaction (qRT-PCR).

15. A method of determining the increased likelihood of having or developing autism in a patient, comprising:
(a) obtaining a physiological sample from the patient;
(b) quantifying levels of GABRρ2 protein and GABRθ mRNA in the sample, and comparing the levels to a control protein sample; and
(c) determining that the patient has autism or an increased likelihood of developing autism based upon the level of GABRρ2 protein in the sample and based on the level of GABRθ mRNA in the sample, wherein a lower level of GABRρ2 protein and a lower level of GABRθ mRNA in the sample as compared to the control indicates that the patient has autism or has an increased likelihood of developing autism.

16. The method of claim 15, wherein the physiological sample is brain tissue, blood or blood product or cerebral spinal fluid (CSF).

17. The method of claim 15, wherein the protein level is determined by means of a Western blot.

18. The method of claim 15, wherein the mRNA level is determined by means of quantitative real-time polymerase chain reaction (qRT-PCR).
